(19) United States
(12) Patent Application Publication
(10) Pub. No.: US 2020/0129389 A1
(43) Pub. Date: Apr. 30, 2020

(54) OIL-IN-WATER EYELASH COSMETIC

(71) Applicant: JO Cosmetics Co., Ltd., Tokyo (JP)

(72) Inventors: Daiki YOSHIHARA, Tokyo (JP); Hiroshi SHIMA, Tokyo (JP)

(73) Assignee: JO Cosmetics Co., Ltd., Tokyo (JP)

(21) Appl. No.: 16/493,462

(22) PCT Filed: Jun. 14, 2018
(86) PCT No.: PCT/JP2018/022667

§ 371 Date: Sep. 12, 2019

(30) Foreign Application Priority Data
Jul. 4, 2017 (JP) 2017-131075

(51) Int. Cl. A61K 8/06; A61K 8/92; A61K 8/86; A61Q 1/10

(52) U.S. Cl. CPC A61K 8/062; A61Q 1/10; A61K 8/86; A61K 8/922

(57) ABSTRACT

An oil-in-water eyelash cosmetic containing (A) 1 to 40% by mass based on solid content of a film-forming polymer emulsion (A) containing an emulsion of a copolymer of ethylenically unsaturated carboxylic acid monomer and styrene monomer (a-1), 0.1 to 10% by mass of a nonionic surfactant having an HLB value of 6 to 12 (B); and (C) 1 to 40% by mass of a wax is provided. The oil-in-water eyelash cosmetic can be readily removed with warm water without using a special remover, while having good properties in water resistance, curling effect and curl-retaining effect.

### TECHNOLOGICAL FIELD

This invention relates to an oil-in-water eyelash cosmetic, and more specifically, relates to an oil-in-water eyelash cosmetic excellent in curling effect and curl retaining effect as well as water resistance and removability by washing with warm water.

### BACKGROUND TECHNOLOGY

An eyelash cosmetic, a representative of which is mascara, has a makeup effect to make eyes striking by curling eyelashes upwards or making the eyelashes thick and long. Conventionally, various types of eyelash cosmetics such as oil-based type, water-in-oil emulsion type, oil-in-water emulsion type and aqueous type have been known. In recent years, a mascara of the oil-in-water emulsion type containing a film forming polymer that can be readily removed with warm water without using a special remover has become popular.

In connection with such oil-in-water emulsion eyelash cosmetic containing a film forming polymer, Non-Patent Document 1 discloses that incorporation of an acrylic polymer emulsion gives a flexible film having good water resistant, while incorporation of a vinyl acetate polymer emulsion gives a film excellent in cleansing property, but inferior in film properties (see page 78, right column). Thus, water resistance and removability with warm water are in a contradictory relationship, and when higher water resistance is required, there is a problem that removability with warm water is reduced.

As an example of the oil-in-water eyelash cosmetic containing a polymer emulsion, Patent Document 1 discloses an oil-in-water eyelash cosmetic that includes an alkyl acrylate/vinyl acetate copolymer emulsion and an alkyl acrylate copolymer emulsion in a specific ratio, and further includes carbon black, a polyhydric alcohol and polyvinyl pyrrolidone or polyvinyl alcohol. The literature discloses that the oil-in-water eyelash cosmetic is excellent in gloss, blackness, cleansing property with lukewarm water, and storage stability (see Abstract). The Examples and Comparative Examples of the literature disclose oil-in-water eyelash cosmetics containing, as a surfactant, a triethanolamine salt of stearic acid which is prepared in situ using stearic acid and triethanolamine, and disclose that an oil-in-water eyelash cosmetic containing an alkyl acrylate/vinyl acetate copolymer emulsion and an alkyl acrylate copolymer emulsion in a specific ratio exhibits good properties, while an oil-in-water eyelash cosmetic containing a polyvinyl acetate emulsion or an acrylic acid/styrene copolymer emulsion in place of the above-mentioned emulsions is insufficient in storage stability, gloss, blackness, and cleansing property with lukewarm water (see Table 1).

Patent Document 2 discloses an eyelash cosmetic containing an anionic surfactant, which is a combination of 2-amino-2-methyl-1,3-propane diol and a long chain fatty acid, and a film-forming polymer within a specific ratio, and discloses that the above composition enables the eyelash cosmetic to be readily removed without bleeding upon cleansing with warm water while having excellent water resistance. In the invention of this literature, use of a specific anionic surfactant and a film forming polymer within a specific ratio is required, and the literature discloses that when an eyelash cosmetic contains the specific anionic surfactant and the film forming polymer outside the ratio or any other anionic surfactant than the specific anionic surfactant, sufficient cleansing property (removability) cannot be obtained (see Table 1). The literature also discloses that the eyelash cosmetic may contain a nonionic surfactant having an HLB value less than 8 or a nonionic surfactant having an HLB value of 8 or more, and discloses a composition containing styrene/acrylate/ammonium methacrylate copolymer as a film forming polymer, 2.5% by weight of sorbitan stearate having an HLB value of 4.7 and 2.0% by weight of steareth-20 having an HLB value of 18 in Example 1 (see Table 1). As a result of experiments in connection with the composition, the present inventors have found that the combination of the film forming polymer with the nonionic surfactant having such an HLB value does not provide an eyelash cosmetic in which water resistance and cleansing property are sufficiently compatible.

### PRIOR ART DOCUMENT

### Patent Document

Patent Document 1: Japanese Unexamined Patent Publication No. 2010-077042 A
Patent Document 2: JP WO 2014/508107 A

### Non-Patent Document

Non-Patent Document 1: Okuda Shinsuke “Progress in product design technology for mascara” Fragrance Journal Special Issue” No. 21, p. 75 to 79, 2009

### DISCLOSURE OF THE INVENTION

### Problem to be Solved by the Invention

The present invention was completed under the aforementioned background art, and an object of the invention is to provide an oil-in-water eyelash cosmetic capable of being readily removed with warm water without using any specific remover while maintaining good water resistance, curling effect and curl retaining effect.

### Means Used to Solve the Problem

As a result of intensive studies to solve the above-mentioned problems, the present inventors have found that use of a nonionic surfactant having an HLB value within a specific range with a certain film-forming polymer emulsion for preparing an oil-in-water eyelash cosmetic results in an eyelash cosmetic that can be readily removed with warm water regardless of having excellent water resistance. The present invention was completed based on the above knowledge.

Then, the present invention provides an oil-in-water eyelash cosmetic containing 1 to 40% by mass based on solid content of a film-forming polymer emulsion (A), 0.1 to 10% by mass of a nonionic surfactant having an HLB value of 6 to 12 (B), and 1 to 40% by mass of a wax (C), wherein the component (A) contains an emulsion of a copolymer of an ethylenically unsaturated carboxylic acid monomer and a styrene monomer (a-1).

### Effect of the Invention

The oil-in-water eyelash cosmetic of the present invention is excellent in removability by washing with warm water in addition to good water resistance, curling effect and curl retaining effect.

### EMBODIMENTS FOR CARRYING OUT THE INVENTION

The oil-in-water eyelash cosmetic of the present invention contains a film-forming polymer emulsion (A), a nonionic surfactant having an HLB value of 6 to 12 (B) and a wax (C) as essential components.

(Film-Forming Polymer Emulsion)

The film-forming polymer emulsion used as the component (A) contains an emulsion of a copolymer of an α, β-ethylenically unsaturated carboxylic acid monomer and a styrene monomer (a-1) as an essential component. The α, β-ethylenically unsaturated carboxylic acid monomer used to produce the copolymer is an unsaturated carboxylic acid derivative, and examples of which include an unsaturated carboxylic acid having 3 to 5 carbon atoms such as acrylic acid, methacrylic acid, crotonic acid, maleic acid, and itaconic acid; its ester, its amide, and its salt. Examples of the ester of unsaturated carboxylic acid include alkyl esters such as methyl ester, ethyl ester, butyl ester, isobutyl ester, 2-ethylhexyl ester, lauryl ester and cyclohexyl ester; aryl esters such as phenyl ester and benzyl ester; hydroxyalkyl esters such as hydroxyethyl ester and 2-hydroxypropyl ester and the like. Examples of the amide of unsaturated carboxylic acid include amide, N-methylamide, N-ethylamide, N-butylamide, N-methylolamide and the like. Examples of the salt of unsaturated carboxylic acid include ammonium salt, sodium salt, potassium salt and the like.

These monomers can be used solely or in combination of two or more monomers as needed. Of these, (meth) acrylic acid monomer such as (meth) acrylic acid, its salt or its alkyl ester are preferably used. Incidentally, the term “(meth) acrylic acid” means acrylic acid, methacrylic acid or a mixture thereof. Meanwhile, examples of the styrene monomer include styrene, α-methyl styrene, vinyl toluene and the like. Of these, styrene is preferably used in particular.

Preferred examples of the component (a-1) is an emulsion of a copolymer obtained by copolymerizing styrene and at least one monomer selected from (meth) acrylic acid and its simple esters. Examples of the emulsion include (styrene/acrylates) copolymer (INCI name: Styrene/Acrylates Copolymer) that is referred to as alkyl acrylate/styrene copolymer emulsion in Japanese Standards of Quasi-drug Ingredients 2006; an emulsion of a copolymer of styrene, ammonium methacrylate and at least one monomer selected from (meth) acrylic acid and its simple esters that is referred to (styrene/acrylates/ammonium methacrylate) copolymer (INCI name; Styrene/Acrylates/Ammonium Methacrylate Copolymer); an emulsion of ammonium salt of a copolymer of at least one monomer selected from acrylic acid, methacrylic acid and simple esters thereof, methyl styrene and styrene that is referred to ammonium salt of (acrylates/methyl styrene/styrene) copolymer (INCI name; Ammonium Acrylates/Methyl Styrene/Styrene Copolymer); and the like.

The component (a-1) of the present invention may be an emulsion of a composite polymer such as a core-shell polymer composed of a copolymer of ethylenically unsaturated carboxylic acid monomer and styrene monomer, and other polymer and/or copolymer. Of these, an emulsion of a core-shell polymer that has a shell portion composed of a copolymer of ethylenically unsaturated carboxylic acid monomer and styrene monomer, and a core portion composed of a (co)polymer of alkyl acrylate is preferred in view of providing good water resistance and good removability by washing with warm water. The core-shell polymer has preferably a ratio by mass of shell to core [Shell/Core] of 50/50 to 97/3.

Examples of commercially available products of (styrene/acrylates) copolymer emulsion include YODOSL GH41F (Akzo Nobel), DAITOSOL 5000STY (Daito Kasei Kogyo Co., Ltd.), and the like. Of these, YODOSOL GH41F is preferably used due to excellent water resistance and curl retaining effect. Examples of commercially available products of (styrene/acrylates/ammonium methacrylate) copolymer emulsion include SYNTRAN5760 (Interpolymer Co., Ltd.), and the like. Examples of commercially available products containing ammonium salt of (acrylates/methyl styrene/styrene) copolymer include EMUPOLY CE-119N (Gifu Shellac Manufacturing Co., Ltd.) having a core portion composed of ethylhexyl acrylate/methyl methacrylate copolymer and a shell portion composed of ammonium salt of acrylates/methyl styrene/styrene copolymer, and a core/shell ratio by mass of 85/15, and the like.

In the present invention, the component (A) may contain an emulsion of alkyl acrylate (co)polymer (a-2) in addition to the component (a-1) that is an essential component of the component (A). Here, the term “alkyl acrylate (co)polymer emulsion” means an emulsion of a homopolymer or copolymer obtained by polymerizing at least one monomer selected from (meth) acrylic acid or its simple esters such as C1 to C8 alkyl esters. Incorporation of the component (a-2) makes it possible to adjust hardness of film, and contributes to enhance cleansing property and separation effect. In the case of using an emulsion of a composite polymer such as a core-shell polymer composed of a polymer corresponding to the component (a-1) and a polymer corresponding to the component (a-2), the emulsion is classified into the component (a-1).

Examples of the alkyl acrylate (co) polymer emulsion include ACLYLATES COPOLYMER (acrylates copolymer) and ACLYLATES/ETHYLHEXYL ACRYLATE COPOLYMER (acrylates/ethylhexyl acrylate copolymer) both of which are named in INCI, i.e., International Nomenclature Cosmetic Ingredient labeling names. Examples of commercially available products regarding the former include YODOSOL GH800F and YODOSOL GH810F both of which are available from Akzo Nobel K.K. Examples of commercially available products regarding the latter include DATOSOL 5000SJ (Daito Kasei Kogyo Co., Ltd.) and the like.

The film-forming polymer emulsion of the component (A) can be usually a commercially available product in which a polymer component is finely dispersed in an aqueous medium within a content of 20 to 60% by mass in terms of solid content. A content of the component (A) is 1 to 40% by mass, preferably 2 to 35% by mass, more preferably 3 to 30% by mass relative to the whole cosmetic in terms of solid content. When the content is excessively small, water resistance, curling effect and curl retaining effect are lowered. While, when the content is excessively large, it becomes difficult to apply a cosmetic due to increased viscosity.

The content of the component (a-1) contained in the film-forming polymer emulsion of the component (A) is 1 to 30% by mass, preferably 2 to 25% by mass, more preferably 3 to 20% by mass relative to the whole cosmetic in terms of solid content. When the content is excessively small, water resistance, curling effect and curl retaining effect are lowered. While, when the content is excessively large, removability by washing with warm water decreases.

The component (A) may consist of the component (a-1) solely. When containing the component (a-2), the ratio by mass of the component (a-2) to the component (a-1) represented by (a-2)/(a-1) is preferably 0.01 to 5, more preferably 0.05 to 3, more preferably from 0.1 to 2 in terms of solid content. By controlling the ratio within the range, hardness of the film can be adjusted, and cleansing property and separation effect can be enhanced. However, when the ratio is excessively large, water resistance tends to decrease.

In the present invention, the component (A) may contain other film-forming polymer emulsions in addition to the component (a-1) and the component (a-2) as long as the effects of the present invention are not substantially impaired. Examples of the other film-forming polymer emulsions include polyvinyl acetate emulsion, alkyl acrylate-vinyl acetate copolymer emulsion, alkyl methacrylate-vinyl acetate copolymer emulsion, polyurethane emulsion, urethane-based copolymer emulsion, and the like. A larger content of the other film-forming polymer emulsions tends to reduce water resistance and removability by washing with warm water. Hence, its content is preferably 20% by mass or less, more preferably 10% by mass or less relative to the total mass of the component (a-1) and the component (a-2) in terms of solid content. In particular, it is preferred that the component (A) does not substantially contain other film-forming polymer emulsions.

(Nonionic Surfactant) In the present invention, a nonionic surfactant is used as the component (B) in order to improve cleansing property. The nonionic surfactant has an HLB value of 6 to 12, preferably 7 to 10. When the HLB value is outside this range, it not possible to improve cleansing property with warm water (removability).

Examples of the nonionic surfactants include polyglycerol fatty acid esters such as polyglyceryl-4 stearate, polyglycerol-10 distearate, polyglyceryl-2 oleate; polyoxyethylene hydrogenated castor oils such PEG-10 hydrogenated castor oil and PEG-20 hydrogenated castor oil; polyoxyethylene fatty acid esters such as PEG-5 stearate, PEG-6 isostearate; polyoxyethylene alkyl ethers such as ceteth-2, oleth-2, steareth-15; fatty acid polyoxyethylene alkyl ethers such as steareth-12 stearate and laureth-10 isostearae; polyoxyethylene fatty acid glyceryl such as PEG-6 glyceryl isostearate, PEG-20 glyceryl triisostearate and PEG-20 glyceryl tristearate; fatty acid polyoxyethylene hydrogenated castor oil such as PEG-20 hydrogenated castor oil isostearate and PEG-20 hydrogenated castor oil triisostearate; polyoxyethylene sorbitan fatty acid ester, polyoxyethylene polyoxypropylene copolymer, ether of polyoxyethylene polyoxypropylene copolymer and long-chain alcohol, ether of polybutylene glycol polyglycerin copolymer and long-chain alcohol, and the like. Of these, polyoxyethylene fatty acid ester, polyoxyethylene fatty acid glyceryl and fatty acid polyoxyethylene alkyl ether are preferably used.

When the nonionic surfactant contains a fatty acid residue in its molecule, it is preferably a residue of a higher fatty acid having 10 to 22 carbon atoms such as myristic acid, palmitic acid, stearic acid, isostearic acid and oleic acid. Examples of commercial products suitably used as the component (B) include EMALEX GWS-320 (PEG-20 glyceryl tristearate; Nihon Emulsion Co., Ltd.; HLB 8), UNIOX GT-20IS (PEG-20 glyceryl isostearate; NOF Corporation; HLB 8), EMALEX PEIS-6EX (PEG-6 isostearate; Nihon Emulsion Co., Ltd.; HLB 9), and EMALEX SWS-12 (steareth-12 stearate; Nihon Emulsion Co., Ltd.; HLB 8).

The component (B) may be used alone or in combination of two or more. A content of the component (B) used is 0.1 to 10% by mass, preferably 0.5 to 8% by mass, and more preferably 1 to 6% by mass in the whole composition. When the content is excessively small, removability with warm water decreases, whereas when it is excessively large, water resistance decreases.

(Wax)

In the present invention, the wax used as the component (C) is a solid oil having a melting point of 50° C. or higher. Examples of the wax include animal wax, vegetable wax, mineral wax, silicone wax, synthetic wax and the like. The wax preferably has a melting point of 50° C. or higher and 110° C. or lower. The melting point of the wax is a value measured according to the second method of the melting point measurement method which is the general test method defined in Japanese Standards of Quasi-drug Ingredients.

Examples of the wax include beeswax, white beeswax, carnauba wax, candelilla wax, spermaceti, montan wax, rice bran wax, lanolin, Japan wax, hexyl laurate, hydrogenated lanolin, hydrogenated jojoba oil, hard lanolin, shellac wax, microcrystalline wax, paraffin wax, polyethylene wax, Fischer-Tropsch wax, hydrogenated oil, hydrogenated castor oil, petrolatum, alkyl silicone, jojoba ester, stearyl alcohol, behenyl alcohol and the like.

Examples of commercially available waxes include bees wax or white beeswax such as SA Bees wax-PA (melting point 60° C. to 67° C.) available from Croda Japan K.K., Golden Brand (melting point 60° C. to 67° C.) and WHITE BEES WAX (melting point 60° C. to 67° C.) available from Miki Chemical Industry & Co., Ltd.; carnauba wax such as Purified Carnauba Wax No. 1 (melting point 80° C. to 86° C.) available from Cerarica Noda Co., Ltd.; micro crystalline wax such as HNP-9 (melting point 74° C. to 78° C.), HI-MIC-2065 (melting point 72° C. to 78° C.), HI-MIC-1070 (melting point 77° C. to 82° C.), Hi-Mic-1080 (melting point 82° C. to 88° C.), HI-MIC-1090 (melting point 86° C. to 90° C.), HNP-0190 (melting point 87° C. to 93° C.) all of which are available from Nippon Seiro Co., Ltd. and MULTIWAX W-445 (melting point 77° C. to 82° C.) available from Sonneborn Co., Ltd.; paraffin wax such as PARAFFIN WAX 135 (melting point 57° C. to 60° C.), PARAFFIN WAX 140 (melting point 60° C. to 63° C.), PARAFFIN WAX 150 (melting point 66° C. to 68° C.) and HNP-11 (melting point 66° C. to 70° C.) available from Nippon Seiro Co., Ltd.; polyethylene wax such as PERFORMALENE 400 (melting point 75° C. to 90° C.), 500 (melting point 83° C. to 92° C.), 655 (melting point 93 to 102° C.) all of which are available from NEW PHASE TECHNOLOGIES; and silicone wax such as SF 1642 (melting point 60° C. to 70° C.) available from Momentive Performance Materials Japan Ltd.

A content of the component (C) is 1 to 40%, preferably 2 to 35%, more preferably 5 to 30% by mass with respect to the whole cosmetic. When the content is excessively small, curling effect and curl retaining effect decrease. When the content is excessively large, a cosmetic becomes sticky due to increase of viscosity, and it becomes difficult to makeup beautifully due to decrease of applicability.

The component (C) preferably contains a wax having a melting point of 70° C. or higher. Examples of such a wax include carnauba wax, microcrystalline wax, polyethylene wax and the like. Of these, carnauba wax is preferably used in particular. Incorporation of the wax having a melting point of 70° C. or higher contributes to enhance water resistance, curling effect and curl retaining effect. A content of the wax having a melting point of 70° C. or higher is preferably 1 to 30% by mass, more preferably 2 to 25% by mass with respect to the whole cosmetic. A ratio by mass of the wax having a melting point of 70° C. or higher to the component (C) is preferably 0.2 to 1, more preferably 0.3 to 0.8.

In the present invention, the oil-in-water eyelash cosmetic may contain a hydrophilic surfactant, which is referred to as component (D), in addition to the above components (A) to (C) in order to homogeneously and stably emulsify oil components such as a wax. The hydrophilic surfactant can be added in accordance with conventional methods such as a method of adding the surfactant in components forming an aqueous phase or oil phase, and a method of adding a higher fatty acid and a basic compound respectively into an oil phase and an aqueous phase followed synthesizing the surfactant in situ by mixing both phases. In the case that the wax used as the oil component contains an effective amount of the higher fatty acid, it is possible to synthesize the surfactant in situ by reacting the higher fatty acid contained in the wax with a basic compound that is separately added. For example, white beeswax having an acid value of 17 to 22 mg/g can produce a higher fatty acid salt by reaction with a basic compound.

Examples of the hydrophilic surfactant that can be used include anionic surfactants, nonionic surfactants, cationic surfactants, amphoteric surfactants and the like. Examples of the anionic surfactant include inorganic or organic salts of fatty acid such as stearic acid and lauric acid, alkylbenzene sulfates, alkyl sulfonates, α-olefin sulfonates, dialkyl sulfosuccinates, a-sulfonated fatty acid salts, acyl methyl taurine salts, N-methyl-N-alkyl taurine salts, polyoxyethylene alkyl ether sulfates, polyoxyethylene alkyl phenyl ether sulfates, alkyl phosphates, polyoxyethylene alkyl ether phosphates, polyoxyethylene alkyl phenyl ether phosphates, N-acylamino acid salts, N-acyl-N-alkylaminoacid salts, alkyl sulfosuccinates, and the like. Of these, an inorganic or organic salt of a higher fatty acid having a carbon number of 12 to 24 and a combination of a natural wax having acid value of 10 or more with an inorganic or organic basic compound are preferably used in view of easiness of emulsification process, stability of emulsion, and less impairment of curling effect. As the natural wax having acid value of 10 or more, beeswax and white beeswax are preferably used.

In the case of using the inorganic salt and/or organic salt of higher fatty acid having 12 to 24 carbon atoms as the hydrophilic surfactant, its content is preferably 0.1 to 10% by mass, more preferably 0.5 to 8% by mass in the whole cosmetic in terms of free acid content. In the case of synthesizing the hydrophilic surfactant in situ by combining a natural wax having an acid value of 10 or more with a basic compound, it is preferred that a content of the resultant higher fatty acid salt is preferably 0.1 to 15% by mass, more preferably 0.5 to 12% by mass. The basic compound is preferably used in an amount of 0.5 to 1.5 times the moles necessary for neutralizing an acid component which is calculated based on an acid value of the natural wax. When using the hydrophilic surfactant produced by the combination of the natural wax having an acid value of 10 or more and the inorganic and/or organic basic compound, more preferable properties are achieved due to good compatibility of the hydrophilic surfactant and the component (C). In the present invention, even if a higher fatty acid contained in the natural wax having an acid value of 10 or more converts to a higher fatty acid salt via reaction with a basic compound, the amount of the natural wax incorporated is deemed to be included in an amount of the component (C).

The hydrophilic nonionic surfactant has an HLB value of more than 12, and examples thereof include glycerol fatty acid esters and alkylene glycol adducts thereof, polyglycerol fatty acid esters and alkylene glycol adducts thereof, propylene glycol fatty acid esters and alkylene glycol adducts thereof, sorbitan fatty acid esters and alkylene glycol adducts thereof, fatty acid esters of sorbitol and alkylene glycol adducts thereof, polyalkylene glycol fatty acid esters, sucrose fatty acid esters, polyoxyalkylene alkyl ethers, glycerol alkyl ethers, polyoxyethylene alkyl phenyl ethers, polyoxyethylene hydrogenated castor oil, alkylene glycol adducts of lanolin, polyoxyalkylene-alkyl co-modified organopolysiloxane, polyether-modified organopolysiloxane and the like. The content of the hydrophilic nonionic surfactant is preferably 5% by mass or less, more preferably 4% by mass or less in view of water resistance.

In the present invention, in addition to the above components (A) to (D), an oil-soluble resin can be blended as component (E). The oil-soluble resin is not particularly limited as long as it is soluble in oily components which form an internal phase. Examples thereof include silicone resin such as trimethylsiloxysilicate, partially crosslinked organopolysiloxane, trimethylsiloxysilylpropylcarbamoyl pullulan, fluorine modified silicone, acrylic modified silicone, silicone dendrimer modified resin compound; rosin acid resin such as pentaerythrityl rosinate; Candelilla resin; polyvinyl acetate type resin; polyvinyl isobutyl ether; polyisobutylene; and the like. Here, Candelilla resin means a resin component obtained by separating and extracting Candelilla wax with an organic solvent, and has a resin content of preferably 65% or more, more preferably 85% or more. Examples of commercially available Candelilla resin include Candelilla resin E-1 available from Japan Natural Products Inc.

In the cosmetic of the present invention, a coloring material can be blended as component (F). The coloring material is not particularly limited by its shape, particle diameter, and particle structure as long as it is commonly used in the field of cosmetics. Examples of the shape include a spherical shape, a plate shape, and a needle shape. Examples of the particle size include an aerosol, a fine particle and a pigment grade. The particle structure may be porous or nonporous. A content of the colorant (F) can be appropriately selected. It is preferably 0.1 to 20%, more preferably 0.5 to 15%, by mass with respect to the whole composition. When the content of the coloring material is excessively large, adhesion tends to decrease. Conversely, when it is excessively small, coloring effect sometimes becomes insufficient.

Examples of the colorant (F) include inorganic powders, glittering powders, organic powders, pigment powders, metal powders, composite powders, and the like. More specifically, there can be mentioned inorganic pigments such as talc, mica, kaolin, calcium carbonate, silica, zinc oxide, titanium dioxide, red iron oxide, yellow iron oxide, black iron oxide, ultramarine blue, iron blue, carbon black, low order titanium oxide, cobalt violet, chromium oxide, chromium hydroxide, cobalt titanate, bismuth oxychloride and titanium-mica-based pearl pigment; resin powders of organic polymer such as polyamide resin, polyethylene resin, polyacryl resin, polyester resin, fluorine resin, cellulose resin, polystyrene resin, copolymer resin such as styrene-acryl copolymer resin, polypropylene resin, silicone resin, urethane resin; organic pigment such as Red No. 201, Red No. 202, Red No. 204, Red No. 205, Red No. 220, Red No. 226, Red No. 228, Red No. 405, Orange No. 203, Yellow No. 205, Yellow No. 4, Yellow No. 5, Blue No. 1, Blue No. 404, Green No. 3, zirconium lake thereof, barium lake thereof or aluminum lake thereof; natural coloring materials such as chlorophyll and β-carotene; dyes and the like. These coloring materials may be used alone or in combination of two or more. Of these, black coloring materials, particularly black iron oxide and carbon black are preferably used.

In addition to the above components, the oil-in-water eyelash cosmetic of the present invention may contain components which are used in conventional cosmetics, for example, additives such as liquid or semi-solid oils having a melting point of less than 50° C., oily gelling agents, water-soluble thickeners, pigment dispersants, polyhydric alcohols, lower alcohols, ultraviolet absorbers, ultraviolet scattering agents, humectants, fragrances, antioxidants, preservatives, sequestering agents, defoaming agents, fibers, dyes, various extracts as long as they do not substantially impair the effect of the present invention.

Examples of the liquid or semi-solid oils having a melting point of less than 50° C. include volatile hydrocarbon oils such as light liquid isoparaffin and isododecane; volatile silicone oils such as decamethylcyclopentasiloxane, octamethylcyclotetrasiloxane, dimethylpolysiloxanes having a low polymerization degree, and methyl trimethicone; hydrocarbon oils such as liquid paraffin, squalane, petrolatum, polybutene, and hydrogenated polyisobutene; fats and oils such as olive oil, castor oil, mink oil, and macadamia nut oil; esters such as cetyl ethylhexanoate, isopropyl myristate, isopropyl palmitate, octyldodecyl myristate, triethylhexanoin, neopentyl glycol dioctanoate, cholesterol fatty acid esters, cholesteryl/behenyl/octyldodecyl lauroyl glutamate, and jojoba oil; higher alcohols such as oleyl alcohol and isostearyl alcohol; silicones such as non-volatile dimethylpolysiloxane, methylphenyl polysiloxane, partially crosslinked organopolysiloxane and fluorine-modified polysiloxane; fluorine type oils such as perfluorodecane, perfluorooctane and perfluoro polysiloxanes; lanolin derivatives such as lanolin, lanolin acetate, lanolin fatty acid isopropyl, and lanolin alcohols; and the like. However, when these liquid oils are contained in a large amount, the water resistance and the curling effect tend to be lowered. Therefore, in the case of containing the liquid oils, the content thereof is preferably 10% or less, more preferably 5% or less, by mass with respect to the whole cosmetic.

Examples of the oily gelling agents include dextrin fatty acid esters, sucrose fatty acid esters, starch fatty acid esters, aluminum stearate, and organically modified clay minerals such as disteardimonium hectorite. The oily gelling agents can be appropriately added to improve stability and usability.

Examples of the water-soluble thickeners or powdery pigment dispersants include bentonite, xanthan gum, cellulose gum, sodium polyacrylate, and polyvinyl pyrrolidone. These thickeners or dispersants can be used alone or in combination of two or more. Of these, xanthan gum is preferably used due to easiness of viscosity control without impairing easiness of coating. The content of the thickener or dispersant is preferably 0.1 to 20% by mass more preferably 0.2 to 10% by mass. When the content is excessively small, it becomes difficult to adjust to sufficient viscosity. While, the content is excessively large, smoothness tends to be insufficient due to higher viscosity.

The oil-in-water eyelash cosmetic of the present invention can be prepared in accordance with conventional methods except using the above-described components. For example, it can be prepared by preparing an aqueous phase (hereinafter sometimes referred to as phase A) and an oil phase (hereinafter sometimes referred to as phase B) respectively, heating and mixing the both phases to make an oil-in-water emulsion, and cooling the resultant emulsion to room temperature. In the case of incorporating a water-soluble compound as an optional component, it is added to the phase A. In the case of incorporating an oily component such as a wax as an optional component, it is added to the phase B. The oil-in-water emulsion can also be prepared by using a hydrophilic surfactant which is produced by adding a basic compound being a raw material of the surfactant to the phase A and a higher fatty acid and/or a natural wax having a specific acid value being another raw material of the hydrophilic surfactant to the phase B followed by slowly adding the phase A to the phase B with stirring to react both raw materials in situ.

The oil-in-water eyelash cosmetic of the present invention can be used as a mascara, a mascara makeup base, a mascara top coat, an eyelash essence and the like. The cosmetic may have dosage forms appropriately selected in accordance with its application. For example, the dosage form can be creamy, liquid or the like. The oil-in-water eyelash cosmetic can be applied to the eyelashes using appropriate tools such as brushes, molding applicators made of plastics or metals, and the like.

In the case of making up eyelashes using the oil-in-water eyelash cosmetic of the present invention, the makeup can be readily removed by using warm water of about 35° C. to 45° C. without using a remover which is usually used for removing a makeup of oil-in-water eyelash cosmetics.

### EXAMPLES

Hereinafter, the present invention will be further described with reference to Examples and Comparative Examples, but the present invention is not limited by these Examples. The content of each ingredient in the formulations in the following description is expressed in % by mass with respect to the whole composition unless otherwise specified. The evaluation methods of the oil-in-water eyelash cosmetic in the following Examples and Comparative Examples are as follows.

(Evaluation Methods a to e)

As to the following evaluation items a to e, ten evaluators applied each sample to their own eyelashes, and scored at seven levels of from 0 to 6 based on the evaluation criteria shown in item (1) below. Using the total score of 10 evaluators, properties as an oil-in-water eyelash cosmetic were evaluated according to the 4-step criterion shown in item (2) below. The water resistance of the evaluation item a and the curl retaining effect were evaluated based on the degree of bleeding of the cosmetic at 6 hours after application, Further, the removability by warm water of 40° C. in the evaluation item b was evaluated by immersing a piece of a commercial cotton into warm water (tap water) of 40° C., sandwiching the eyelashes with a sample applied thereto by the cotton for 30 seconds to be in contact with warm water, wiping off the sample, and visually observing a residual amount of the sample on the eyelashes.

(Evaluation Item)

a. Water resistance (normal temperature)

b. Removability by warm water of 40° C.

c. Easiness of applicability

d. Curling effect

e. Curl retaining effect

(1) Evaluation criteria

(Rating): (Evaluation)

6: Very good

5: Good

4: Slightly good

3: Average

2: Somewhat bad

1: Bad

0: Very bad

(2) Four-grade criteria

(Evaluation): (Total of Evaluation Score)

⊚: Total point ranges from 46 to 60

◯: Total point ranges from 31 to 45

Δ: Total point ranges from 16 to 30

x: Total point ranges from 0 to 15

### Examples 1 to 4 and Comparative Examples 1 to 3

### <Oil-in-Water Mascara>

Mascaras of the formulation shown in Table 1 were prepared according to the following production procedure, and as to water resistance at normal temperature, easiness of removal by warm water, easiness of applicability, curling effect and curl retaining effect, sensory evaluations were carried out according to the foregoing method. The results are also shown in Table 1.

(Production Procedure)

(1) Ingredients for Phase A shown in Table 1 were heated to about 85° C. and mixed to prepare Phase A being an aqueous phase.
(2) Ingredients for Phase B shown in Table 1 were heated to about 95° C. and mixed to prepare Phase B being an oil phase.
(3) Using a disper mixer, Phase B was mixed with Phase A to prepare an emulsion which was made using a higher fatty acid salt synthesized in situ via neutralization of a carboxylic acid group contained in white beeswax with an amino methyl propanol and a hydrophilic nonionic surfactant contained in Phase A.
(4) After cooling the emulsion to 60° C. with stirring, ingredients of Phase C were added to the emulsion, and the resultant mixture was homogeneously mixed using a disper mixer.
(5) The emulsion in a homogeneous state was cooled to 32° C. to obtain an oil-in-water mascara.

TABLE 1

| | | Example | | | | Comparative Example | | |
|---|---|---|---|---|---|---|---|---|
| Phase | Component | 1 | 2 | 3 | 4 | 1 | 2 | 3 |
| A | Purified water | Balance | Balance | Balance | Balance | Balance | Balance | Balance |
| | Dipropylene glycol | 4.5 | 4.5 | 4.5 | 4.5 | 4.5 | 4.5 | 4.5 |
| | 1,3-butylene glycol | 2.2 | 2.2 | 2.2 | 2.2 | 2.2 | 2.2 | 2.2 |
| | Amino methyl propanol | 0.3 | 0.3 | 0.3 | 0.3 | 0.3 | 0.3 | 0.3 |
| | 1,2-pentanediol | 2.5 | 2.5 | 2.5 | 2.5 | 2.5 | 2.5 | 2.5 |
| | Polyvinyl pyrrolidone | 1 | 1 | 1 | 1 | 1 | 1 | 1 |
| | Xanthan gum | 1.4 | 1.4 | 1.4 | 1.4 | 1.4 | 1.4 | 1.4 |
| | D-pantothenyl alcohol | 0.1 | 0.1 | 0.1 | 0.1 | 0.1 | 0.1 | 0.1 |
| | Polyoxyethylene (20) sorbitan | 1.5 | 1.5 | 1.5 | 1.5 | 1.5 | 1.5 | 1.5 |
| | monostearate (HLB: 14.9) (*1) | | | | | | | |
| | Polyoxyethylene (20) cetyl ether (HLB: | 0.6 | 0.6 | 0.6 | 0.6 | 0.6 | 0.6 | 0.6 |
| | 17) (*2) | | | | | | | |
| | Carbon black | 1 | 1 | 1 | 1 | 1 | 1 | 1 |
| | Boron nitride | 2 | 2 | 2 | 2 | 2 | 2 | 2 |
| | Talc | 6 | 6 | 6 | 6 | 6 | 6 | 6 |
| B | Petrolatum | 0.5 | 0.5 | 0.5 | 0.5 | 0.5 | 0.5 | 0.5 |
| | Dimethylpolysiloxane (100,000 mm²/s) | 0.9 | 0.9 | 0.9 | 0.9 | 0.9 | 0.9 | 0.9 |
| | Sorbitan monostearate (HLB: 4.7) (*3) | 0.4 | 0.4 | 0.4 | 0.4 | 0.4 | 0.4 | 0.4 |
| | White beeswax (melting point 60-67° C., | 5.4 | 5.4 | 5.4 | 5.4 | 5.4 | 5.4 | 5.4 |
| | acid value 17-22) (*4) | | | | | | | |
| | Carnauba wax (melting point 80~86° C.) (※5) | 8 | 8 | 8 | 8 | 8 | 8 | 8 |
| | Gandelilla resin | 1 | 1 | 1 | 1 | 1 | 1 | 1 |
| | PEG-20 glyceryl tristearate (HLB: 8) (*6) | 2 | 1 | 2 | 3 | — | 2 | 2 |
| C | (Styrene/Acrylates) copolymer (*7) | 17 | 30 | 30 | 30 | 30 | — | — |
| | Acrylates copolymer (*8) | 11 | — | — | — | — | 30 | — |
| | (Acrylates/ethylhexyl acrylate) | — | — | — | — | — | — | 30 |
| | copolymer (*9) | | | | | | | |
| | Phenoxyethanol | 0.5 | 0.5 | 0.5 | 0.5 | 0.5 | 0.5 | 0.5 |
| | Nylon staple fiber (2 mm, 6 denier) | 0.3 | 0.3 | 0.3 | 0.3 | 0.3 | 0.3 | 0.3 |
| Evaluation | Water resistance | ∘ | ⊚ | ⊚ | ∘ | ⊚ | Δ | Δ |
| | Removability by warm water | ⊚ | ∘ | ∘ | ⊚ | x | ⊚ | ⊚ |
| | Easiness of application | ⊚ | ∘ | ∘ | ⊚ | ∘ | ∘ | ∘ |
| | Curling efect | ∘ | ⊚ | ⊚ | ∘ | ∘ | Δ | Δ |
| | Curl-retaining effect | ∘ | ⊚ | ⊚ | ∘ | ∘ | Δ | Δ |

(*1) Product Name: RHEODOL TW-S120V (Kao Corporation): Solid nonionic surfactant having an HLB value of 14.7
(*2) Product Name: NIKKOL BC-20TX (Nikko Chemicals Co.): Solid nonionic surfactant having an HLB value of 17
(*3) Product Name: RHEODOL SP-S10V (Kao Corporation): Solid nonionic surfactant having an HLB value of 4.7
(*4) Product Name: WHITE BEES WAX (Miki Chemical Industry Co., Ltd.): Wax having an acid value of 17 to 22
(※5) Product name: PURIFIED CARNAUBA WAX No. 1 (Cerarica Noda Co., Ltd.)
(*6) Product name: EMALEX GWS-320 (Nihon Emulsion Co., Ltd.): Solid nonionic surfactant having an HLB value of 8
(*7) Product name: YODOSOL GH41F (Akzo Nobel K.K.): Solid content of 45%
(*8) Product name: YODOSOL GH810F (Akzo Nobel K.K.): Solids content of 46%
(*9) Product name: DAITOSOL 50005J (DAITO KASEI KOGYO Co., Ltd.): Solid content of 50%

As is apparent from the results shown in Table 1, the mascaras of Examples 1 to 4 are superior in water resistance, curling effect and curl retaining effect as compared with the mascaras of Comparative Examples 1 to 3, and they can be removed with warm water without using a special remover for a mascara. In particular, the mascara of Example 1 which contains (styrene/acrylates) copolymer (a-1) and acrylates copolymer (a-2) in combination as the component (A) is excellent in removability and easiness of applicability. The mascara of Example 3 which contains an increased amount of nonionic surfactant corresponding to the component (B) is also excellent in removability and easiness of applicability regardless of containing only (styrene/acrylates) copolymer corresponding to the component (a-1) as the component (A). On the other hand, the mascara of Comparative Example 1 not containing the component (B) is inferior in removability, and the mascaras of Comparative Examples 2 and 3 which contain acrylates copolymer or (acrylates/ethylhexyl acrylate) copolymer corresponding to the component (a-2) in place of (styrene/acrylates) copolymer corresponding to the component (a-1) are unsatisfactory in water resistance, curling effect and curl retaining effect although being excellent in removability.

### Examples 5 to 8 and Comparative Examples 4 to 5

### <Oil-in-Water Mascara>

Mascaras of the formulation shown in Table 2 were prepared by the same production procedure as described above, and water resistance at normal temperature, easiness of removal by warm water, easiness of application, curling effect and curl retaining effect were evaluated in accordance with the above evaluation methods. The results are shown in Table 2 together with the formulation.

TABLE 2

| | | | | | | Comparative | |
|---|---|---|---|---|---|---|---|
| | | Example | | | | Example | |
| Phase | Component | 5 | 6 | 7 | 8 | 4 | 5 |
| A | Purified water | Balance | Balance | Balance | Balance | Balance | Balance |
| | Dipropylene glycol | 4.5 | 4.5 | 4.5 | 4.5 | 4.5 | 4.5 |
| | 1,3-butylene glycol | 2.2 | 2.2 | 2.2 | 2.2 | 2.2 | 2.2 |
| | Amino methyl propanol | 0.3 | 0.3 | 0.3 | 0.3 | 0.3 | 0.3 |
| | 1,2-pentanediol | 2.5 | 2.5 | 2.5 | 2.5 | 2.5 | 2.5 |
| | Polyvinyl pyrrolidone | 1 | 1 | 1 | 1 | 1 | 1 |
| | Xanthan gum | 1.4 | 1.4 | 1.4 | 1.4 | 1.4 | 1.4 |
| | D-pantothenyl alcohol | 0.1 | 0.1 | 0.1 | 0.1 | 0.1 | 0.1 |
| | Polyoxyethylene (20) sorbitan monostearate | 1.5 | 1.5 | 1.5 | 1.5 | 1.5 | 1.5 |
| | (HLB: 14.9) (※1) | | | | | | |
| | Polyoxyethylene (20) cetyl ether (HLB: 17) (*2) | 0.6 | 0.6 | 0.6 | 0.6 | 0.6 | 0.6 |
| | Carbon black | 1 | 1 | 1 | 1 | 1 | 1 |
| | Boron nitride | 2 | 2 | 2 | 2 | 2 | 2 |
| | Talc | 6 | 6 | 6 | 6 | 6 | 6 |
| B | Petrolatum | 0. 5 | 0. 5 | 0. 5 | 0. 5 | 0. 5 | 0. 5 |
| | Dimethylpolysiloxane (100,000 mm²/s) | 0.9 | 0.9 | 0.9 | 0.9 | 0.9 | 0.9 |
| | Sorbitan monostearate (HLB:4. 7) (*3) | 0.4 | 0.4 | 0.4 | 0.4 | 0.4 | 0.4 |
| | White beeswax (melting point 60-67° C., acid | 5.4 | 5.4 | 5.4 | 5.4 | 5.4 | 5.4 |
| | value 17-22) (*4) | | | | | | |
| | Carnauba wax (melting point 8~86° C.) (*5) | 8 | 8 | 8 | 8 | 8 | 8 |
| | Candelilla resin | 1 | 1 | 1 | 1 | 1 | 1 |
| | PEG-4 glyceryl tristearate (HLB: | — | — | — | — | 2 | — |
| | 2) (*10) | | | | | | |
| | PEG-20 glyceryl tristearate (HLB: | 2 | — | — | — | — | — |
| | 8) (*6) | | | | | | |
| | Steareth-12 stearate (HLB: | — | 2 | — | — | — | — |
| | 8) (*12) | | | | | | |
| | PEG-20 glyceryl tristearate (HLB: | — | — | 2 | — | — | — |
| | 8) (*13) | | | | | | |
| | Steareth-15 (HLB: 12) (*14) | — | — | — | 2 | — | — |
| | PoIysorbate80 (HLB: 15) (*15) | — | — | — | — | — | 2 |
| C | (Styrene/Acrylates) | 30 | 30 | 30 | 30 | 30 | 30 |
| | copoIymer (*7) | | | | | | |
| | Phenoxyethanol | 0.5 | 0.5 | 0.5 | 0.5 | 0.5 | 0.5 |
| | Nylon staple fiber (2 mm, 6 denier) | 0.3 | 0.3 | 0.3 | 0.3 | 0.3 | 0.3 |
| Evaluation | Water resistance | ∘ | ∘ | ∘ | ∘ | ⊚ | x |
| | Removability by warm water | ⊚ | ⊚ | ⊚ | ⊚ | Δ | ⊚ |
| | Easiness of application | ∘ | ∘ | ∘ | ∘ | ∘ | ∘ |
| | Curling efect | ∘ | ∘ | ∘ | ∘ | ∘ | Δ |
| | Curl-retaining effect | ∘ | ∘ | ∘ | ∘ | ∘ | Δ |

(*10) Product name: EMALEX GWS-304 (Nihon Emulsion Co., Ltd.): Solid nonionic surfactant having an HLB value of 2
※11 Product name: EMALEX RWIS-320 (Nihon Emulsion Co., Ltd.): Liquid nonionic agent having an HLB value of 6
(*12) Product name: EMALEX SWS-12 (Nihon emulsion Co., Ltd.): Solid nonionic surfactant having an HLB value of 8
(*13) Product name: UNIOX GT-20I5 (NOF Corporation): Liquid nonionic surfactant having an HLB value of 8
(*14) Product name: EMALEX 615 (Nihon emulsion Co., Ltd.): Solid nonionic surfactant having an HLB value of 12
(*15) Product name: RHEODOL TW-O 120V (Kao Corporation): Liquid nonionic surfactant having an HLB value of 15

As is apparent from the results shown in Table 2, the mascaras of Examples 5 to 8 are excellent in water resistance, curling effect, curl retaining effect and removability by warm water. On the other hand, the mascara of Comparative Example 4 containing a nonionic surfactant having an HLB value of 2 is insufficient in removability by warm water, and the mascara of Comparative Example 5 containing a nonionic surfactant having an HLB value of 15 is very insufficient in water resistance, and also inferior in curling effect and curl retaining effect.

### Examples 9 to 11

### <Oil-in-Water Mascara>

Mascaras of the formulation shown in Table 3 were prepared by the same production procedure as described above. In Example 9, (styrene/acrylates/ammonium methacrylate) copolymer was used as the component (A). In Example 10, a core-shell polymer having 15% of core polymer being (ethylhexyl acrylate/methyl methacrylate) copolymer and 85% of shell polymer being ammonium salt of (acrylates/methyl styrene/styrene) copolymer was used as the component (A). In Example 11, microcrystalline wax having a melting point of 86 to 90° C. was used in place of carnauba wax used in Example 3. As to water resistance at normal temperature, easiness of removal by warm water, easiness of applicability, curling effect and curl retaining effect, the mascaras were evaluated in accordance with the above evaluation methods. The results are shown in Table 3 together with the formulation.

TABLE 3

| | | Example | | |
|---|---|---|---|---|
| Phase | Component | 9 | 10 | 11 |
| A | Purified water | Balance | Balance | Balance |
| | Dipropylene glycol | 4.5 | 4.5 | 4.5 |
| | 1,3-butylene glycol | 2.2 | 2.2 | 2.2 |
| | Amino methyl propanol | 0.3 | 0.3 | 0.3 |
| | 1,2-pentanediol | 2.5 | 2.5 | 2.5 |
| | Polyvinyl pyrrolidone | 1 | 1 | 1 |
| | Xanthan gum | 1.4 | 1.4 | 1.4 |
| | D-pantothenyl alcohol | 0.1 | 0.1 | 0.1 |
| | Polyoxyethylene (20) sorbitan monostearate | 1.5 | 1.5 | 1.5 |
| | (HLB: 14.9) (*1) | | | |
| | Polyoxyethylene (20) cetyl ether (HLB: 17) (* 2) | 0.6 | 0.6 | 0.6 |
| | Carbon black | 1 | 1 | 1 |
| | Boron nitride | 2 | 2 | 2 |
| | Talc | 6 | 6 | 6 |
| B | Petrolatum | 0.5 | 0.5 | 0.5 |
| | Dimethylpolysiloxane (100,000 mm²/s) | 0.9 | 0.9 | 0.9 |
| | Glycerine monostearate (HLB:3.5) | 0.4 | 0.4 | 0.4 |
| | White beeswax (melting point 60-67° C., acid | 5.4 | 5.4 | 5.4 |
| | value 17-22) (*4) | | | |
| | Microcrystaline wax (melting poin 86~90° C.) | — | — | 8 |
| | (*16) | | | |
| | Carnauba wax (melting point 80~86° C.) (*5) | 8 | 8 | — |
| | Candelilla resin | 1 | 1 | 1 |
| | PEG-20 glyceryl tristearate (HLB:8) | 2 | 2 | 2 |
| | (*6) | | | |
| C | (Styrene/Acrylates) copolymer (*7) | — | — | 30 |
| | (Styrene/Acrylates/Ammonium | 30 | — | — |
| | methacrylate) copolymer (*17) | | | |
| | Core-shell polymer composed of (Ethlhexyl | — | 30 | — |
| | acrylate/Methyl matacrylate) copolymer and | | | |
| | ammonium salt of (Acrylates/Methystyrene/ | | | |
| | Styrene) copolymer (*18) | | | |
| | Phenoxyethanol | 0.5 | 0.5 | 0.5 |
| | Nylon staple fiber (2 mm, 6 denier) | 0.3 | 0.3 | 0.3 |
| Evaluation | Water resistance | ◯ | ⊚ | ◯ |
| | Removability by warm water | ⊚ | ⊚ | ⊚ |
| | Easiness of application | ◯ | ◯ | ◯ |
| | Curling efect | ◯ | ◯ | ◯ |
| | Curl-retaining effect | ◯ | ◯ | ◯ |

※16 Product name: HI-MIC-1090 (Nippon Seiro Co., Ltd.)
※17 Product names: SYNTRAN 5760 (Interpolymer Co., Ltd.): Solid content of 41%
※18 Product name: EMUPOLY CE-119N (Gifu Shellac Manufacturing Co., Ltd.): Solid content of 47%

As is apparent from the results shown in Table 3, the mascaras of Examples 9 to 11 are superior in removability by warm water though curling effect and curl retaining effect are slightly lowered as compared with that of Example 3. In particular, the mascara of Example 10 containing the core-shell polymer emulsion is excellent in both water resistance and removability by warm water.

### INDUSTRIAL APPLICABILITY

The present invention provides an oil-in-water eyelash cosmetic having excellent removability by warm water, curling effect and curl retaining effect in addition to excellent long-lasting performance of cosmetic effect such as water resistance.

1. An oil-in-water eyelash cosmetic comprising 1 to 40% by mass based on solid content of an emulsion of a film-forming polymer (A), 0.1 to 10% by mass of a nonionic surfactant having an HLB value of 6 to 12 (B), 1 to 40% by mass of a wax (C), wherein the component (A) contains an emulsion of a copolymer of an ethylenically unsaturated carboxylic acid monomer and a styrene monomer (a-1).

2. The oil-in-water eyelash cosmetic according to claim 1, wherein the component (a-1) is an emulsion of (styrene/acrylates) copolymer, (styrene/acrylates/ammonium methacrylate) copolymer or ammonium salt of (acrylates/methylstyrene/styrene) copolymer.

3. The oil-in-water eyelash cosmetic according to claim 1, wherein the component (A) contains the component (a-1) and an emulsion of alkyl acrylate (co) polymer (a-2), and a ratio by mass of the component (a-2) to the component (a-1) based on solid content represented by formula [(a-2)/(a-1)] is 0.01 to 5.

4. The oil-in-water eyelash cosmetic according to claim 3, wherein the alkyl acrylate (co)polymer is ACRYLATES COPOLYMER or ACRYLATES/ETHYLHEXYL ACRYLATE COPOLYMER.

5. The oil-in-water eyelash cosmetic according to claim 1, further containing a hydrophilic surfactant (D).

6. The oil-in-water eyelash cosmetic according to claim 5, wherein the hydrophilic surfactant is a salt of higher fatty acid or a nonionic surfactant having an HLB value more than 12.

7. The oil-in-water eyelash cosmetic according to claim 1, wherein the component (a-1) is an emulsion of a core-shell polymer having a shell portion containing a copolymer of ethylenically unsaturated carboxylic acid monomer and styrene monomer.

8. The oil-in-water eyelash cosmetic according to claim 1, wherein the component (C) contains a wax having a melting point of 50 to 110° C.

9. The oil-in-water eyelash cosmetic according to claim 1, wherein the component (C) contains a wax having a melting point of 70° C. or higher, and a ratio by mass of the wax relative to the total mass of the component (C) is 0.2 to 1.

10. The oil-in-water eyelash cosmetic according to claim 9, wherein the wax having a melting point of 70° C. or higher is carnauba wax.

11. The oil-in-water eyelash cosmetic according to claim 1, wherein the component (B) is at least one selected from the group consisting of polyoxyethylene fatty acid ester, polyoxyethylene fatty acid glyceryl and fatty acid polyoxyethylene alkyl ether.

12. The oil-in-water eyelash cosmetic according to claim 6, wherein the salt of higher fatty acid is at least one of an inorganic salt of higher fatty acid having carbon atoms of 12 to 24 or an organic salt of higher fatty acid having carbon atoms of 12 to 24.

13. The oil-in-water eyelash cosmetic according to claim 1, further containing a colorant (F).

14. The oil-in-water eyelash cosmetic according to claim 1, further containing at least one of a water-soluble thickener or a pigment dispersing agent.

15. The oil-in-water eyelash cosmetic according to claim 1, wherein when a liquid oil is contained, a content of the liquid oil is 10% by mass or less.
